(19) United States
(12) Patent Application Publication
(10) Pub. No.: US 2021/0405048 A1
(43) Pub. Date: Dec. 30, 2021

(54) Diagnosing Sepsis or Bacteremia by Detecting Peptidoglycan Associated Lipoprotein (PAL) in Urine

(71) Applicants: Lea Michel, Rochester, NY (US); Judith Hellman, San Francisco, CA (US)

(72) Inventors: Lea Michel, Rochester, NY (US); Judith Hellman, San Francisco, CA (US)

(73) Assignee: Rochester Institute of Technology, Rochester, NY (US)

(21) Appl. No.: 17/292,224

(22) PCT Filed: Nov. 8, 2019
(86) PCT No.: PCT/US2019/060530

§ 371 Date: May 7, 2021

Related U.S. Application Data
(60) Provisional application No. 62/757,211, filed on Nov. 8, 2018.

(51) Int. Cl. G01N 33/569; G01N 33/543

(52) U.S. Cl. CPC G01N 33/56916; G01N 2800/26; G01N 2333/4722; G01N 33/54386

(57) ABSTRACT

A method, device and kit for detecting sepsis or bacteremia in a patient includes detecting peptidoglycan associated lipoprotein (Pal) from Gram-negative bacteria in the urine of the patient is disclosed.

### CROSS REFERENCE

This application claims the benefit of the filing date of U.S. Provisional Patent Application Ser. No. 62/757,211, filed Nov. 8, 2018, which is hereby incorporated by reference in its entirety.

### FIELD

The present disclosure relates to a method, device and kit for detecting sepsis or bacteremia in a patient, and in particular for detecting septic or bacteremic levels of Gram-negative bacteria in a patient, by detecting peptidoglycan associated lipoprotein (Pal) from Gram-negative bacteria in the urine of the patient.

### BACKGROUND

Sepsis is a leading cause of death in hospitals, with Gram-negative sepsis (GNS) accounting for ˜40% of the overall cases. In 2011, sepsis-related medical costs were estimated to be $20 billion, making it the most expensive condition treated in US hospitals. Despite decades of research for various treatments, sepsis remains a leading cause of death in hospitals. The initial bacterial infection and the release of bacterial components stimulate a series of immunological responses, including the release of a wide array of proinflammatory cytokines. Sepsis occurs when host proinflammatory immune responses become abnormally elevated. In severe cases, sepsis can result in organ failure and death.

Lipopolysaccharide (LPS, endotoxin) is one of the bacterial components released from Gram-negative bacteria and has been shown to play a major role in the induction of sepsis. An early seminal study showed that, in humans, polyclonal antisera raised against heat-killed Escherichia coli (E. coli) J5 (featuring an exposed LPS core) reduced death by GNS in half. Subsequent studies showed that antibodies to the LPS core alone were not protective. Later, IgG in J5 antisera was shown to bind three E. coli outer membrane proteins: Lpp, OmpA, and peptidoglycan-associated lipoprotein (Pal). Since those studies, results from in vitro and in vivo experiments have further implicated Pal in the pathology of GNS.

All three of the above OMPs were found to be released from GN bacteria, in complex with LPS, when exposed to human serum and in the blood of burned rats with E. coli 018K+ sepsis. One of the three OMPs (18 kDa) was also detected separately (not in complex) in the blood of burned rats, later identified as Pal.

Pal is highly conserved among Enterobacteriaceae, but can be found in most Gram-negative bacteria. E. coli Pal was shown to be released into the blood of mice in a cecal ligation and puncture (CLP) model of polymicrobial sepsis and to activate macrophages and splenocytes in vitro, and stimulate the production of cytokines in LPS nonresponsive (C3H/HeJ) mice. The same study also showed that E. coli variants with mutant or truncated Pal were less virulent than wild-type bacteria. A Pal-deficient strain of E. coli (with reduced levels of Pal) increased survival from 7% (wild-type E. coli strain) to 33%; a Pal nonsense strain of E. coli (with a truncated version of Pal) further increased survival to 100%, suggesting that Pal itself might be toxic. Purified Pal was injected into C3H/HeJ mice, which resulted in increased levels of TNF-α, IL-6, and IL-1β levels in mouse serum. In addition, Pal greatly increased mortality at the 96 hour (hr.) time point (carrier: 0%; 100 μg: ˜92%). Further, Pal activated inflammation through TLR2, and Pal and LPS synergistically activated macrophages. A more recent study corroborated these findings by demonstrating that Pal from Gram-negative Burkholderia cenocepacia (Bcc) was a significant driver of inflammation (stimulating cytokine secretion); Pal from Bcc was also shown to contribute to virulence and cell adhesion. Taken together, these reports suggest that, in addition to LPS, Pal is released from E. coli and may be a mediator of GNS.

Currently, there is no single test method to fully and accurately diagnose sepsis. However, there are 3 tests that are often used in conjunction to diagnose a sepsis infection. The first method is the only FDA approved method for diagnosing sepsis. Procalcitonin (PCT) is the precursor to the hormone calcitonin. Studies have shown that levels of PCT are elevated in patients with sepsis. PCT is produced in many parts of the body, not just the infected area, and is considered one of the body's systematic responses to a sepsis infection. The FDA has approved a commercially available PCT assay that is used to detect PCT in the urine of patients. This assay yields a mean sensitivity of 77%, and can differentiate between sepsis caused by Gram-negative and Gram-positive bacteria, as well as distinguish between sepsis and Systemic Inflammatory Response Syndrome (SIRS). The major limitation of the PCT test is that although PCT is closely associated with inflammation, it is not yet known whether or not it is specific to inflammation due to infection. There is evidence suggesting that a patient may have amounts of PCT in their urine (especially trauma patients), in the absence of an infection. Because of this, the PCT test is rarely the only method used for sepsis diagnosis.

The second method of sepsis diagnosis is also FDA approved, but is not a method specifically for testing for sepsis infections. Sepsis infections may quickly evolve into septic shock. Symptoms of septic shock include micro- and macro-circulatory dysfunction, arterial hypotension, and decreased delivery of oxygen and nutrients into peripheral tissues. Lactate levels are used to signal organ failure, a symptom of septic shock. Many studies have been performed to correlate lactate levels and mortality rates of sepsis patients. Monitoring the lactate levels in sepsis patients is recommended as a way to measure whether or not the administered antibiotics are working. The limitations of the lactate test are that there are many other disorders that can cause a spike in lactate levels in the blood, including cardiac arrest, seizure, trauma, and excessive muscle activity. This suggests that lactate levels alone are not sufficient to diagnose a sepsis infection.

The third test used for sepsis diagnosis involves measuring white blood cell counts. This method is used in conjunction with the other two tests, as it is the least indicative of infection, and can often result in a false positive diagnosis.

In summary, sepsis can be very difficult to diagnose, which is why multiple approaches are often employed in hospitals (and these approaches can vary between hospitals). One related commercial product is the detection device, which allows for detection of Pal from the Legionella bacteria. A rapid point of care assay is currently used in hospitals to detect the Pal protein from Legionella bacteria in urine to diagnose Legionnaires' disease. This test, however, is not currently used for diagnosis of sepsis.

### SUMMARY

In accordance with one aspect of the present invention, there is provided a method for detecting/diagnosing sepsis or bacteremia caused by Gram-negative bacteria, including: obtaining the urine of a human patient; exposing the urine to a Pal-specific binding agent; and detecting Pal from a Gram-negative bacterium bound to the binding agent.

In accordance with another aspect of the present disclosure, there is provided a device including: a test window and optionally, a control window; an absorbent strip; an immunoassay strip, which contains the Pal-specific binding agent and optionally, a second binding agent to detect creatinine or another control; a container that houses the strips; and a cap to cover the absorbent strip.

In accordance with another aspect of the present disclosure, there is provided a kit including: a device which comprises a test window and optionally, a control window, an absorbent strip, an immunoassay strip, which contains the Pal-specific binding agent and optionally, a second binding agent to detect creatinine or another control, a container that houses the strips, and a cap to cover the absorbent strip; a sterile wipe and cup for clean catch urine collection; and a syringe and filter for optional removal of whole bacterial cells.

In accordance with another aspect of the present disclosure, there is provided a method for detecting Gram-negative bacterial infection in a human, including: bringing a urine sample into contact with at least one detection agent that specifically binds to a Gram-negative Pal sensing target molecule and/or a Gram-negative Pal sensing-associated target molecule under conditions that enable binding of the target molecule with the at least one detection agent; and verifying whether a target molecule has bonded with the at least one detection agent.

These and other aspects of the present disclosure will become apparent upon a review of the following detailed description and the claims appended thereto.

### BRIEF DESCRIPTION OF THE DRAWINGS

FIG. 1 shows a Pal immunoblot of urine in accordance with the present disclosure; and

FIG. 2 shows an anti-Pal immunoblot of urine in accordance with the present disclosure.

### DETAILED DESCRIPTION

The present disclosure relates to a method, device and kit for detecting sepsis or bacteremia in a patient. The method includes detecting peptidoglycan associated lipoprotein (Pal) from Gram-negative bacteria in the urine of the patient.

In an embodiment, a method for detecting septic or bacteremic levels of Gram-negative bacteria in a patient, includes: obtaining the urine of a human patient; optionally, filtering out whole cell bacteria from the urine; exposing the urine to a Gram-negative peptidoglycan associated lipoprotein (Pal) specific binding agent; and detecting the Gram-negative peptidoglycan associated lipoprotein (Pal) bound to the binding agent.

Gram-negative bacteria containing Pal include the following: Escherichia coli and all other Enterobacteriaceae; Haemophilus influenzae; Chlamydia pneumoniae; Helicobacter; Pseudomonas; Moraxella catarrhalis; Leptospira interrogans; Cupriavidus; Thermococcus kodakarensis; Corynebacterium glutamicum; Listeria inoocua; Legionella; Fluoribacter; Tatlockia; Gammaproteobacteria; Halomonas; Chromohalobacter; Plasticicumulans; Methylobacter; Cobetia; Halotalea; Neptuniibacter; Oceanospirillaceae; Solimonas; Halovibrio; Salinisphaera; Kushneria; Ketobacter; Alcanivoracaceae; Xanthomonadales; candidatus; Salinisphaera; Aquicella; Wohlfahrtiimonas; Coxiellaceae; Xenorhabdus; Thiolapillus; Paracoccus; Ewingella americana; Serratia. These Gram-negative bacteria all contain a known and identified peptidoglycan associated lipoprotein (Pal) that is similar in sequence and/or structure to other peptidoglycan associated lipoproteins, including peptidoglycan associated lipoprotein (Pal) from E. coli.

E. coli peptidoglycan associated lipoprotein (Pal) has been shown to be released from the bacterium under certain conditions, such as in the presence of human serum. Peptidoglycan associated lipoprotein (Pal) released by E. coli can be found in the urine of patients with E. coli sepsis. Therefore, it is reasonable to expect that peptidoglycan associated lipoprotein (Pal) from other Gram-negative bacteria behave in a similar manner when exposed to human serum. That is, when a person is infected with a Gram-negative bacterium that contains peptidoglycan associated lipoprotein (Pal), that peptidoglycan associated lipoprotein (Pal) is likely to be released by the bacterium and filtered into that person's urine for excretion, thus allowing for detection of peptidoglycan associated lipoprotein (Pal) in that person's urine in accordance with the present methods.

A binding agent that is specific for Pal from one or more Gram-negative bacteria(um) can be prepared according to the following. Such a binding agent can be obtained by understanding the primary sequence of the Pal protein and/or the tertiary structure of the Pal protein and/or producing the Pal protein using known recombinant protein expression methods or native purification methods. Once a purified Gram-negative Pal is obtained, animals could be with immunized the purified protein to obtain a monoclonal or polyclonal antibody specific for Pal. As an example, a monoclonal antibody (6D7) was produced in mice. That monoclonal antibody binds specifically to Pal from E. coli, and cross-reacts with Pal from any Enterobacteriaceae. After immunizing mice with the purified E. coli Pal protein, the spleens were harvested from those mice to obtain B cells. Those B cells were fused with immortal B cells to produce hybridoma cells, which produced the 6D7 monoclonal antibody, which can be used as a binding agent.

In cases where patients are catheterized, one can obtain urine from the drainage bag; in cases where patients are not catheterized, urine will be obtained using normal clean catch methods collected in a sterile cup. Optionally, urine may be filtered to remove whole cell bacteria using a syringe and 0.45 μm attached filter. Total volume required will vary depending on the specific detection test, but 5-10 mL would be a suitable amount.

In accordance with the procedure, the urine is exposed to a Gram-negative peptidoglycan associated lipoprotein (Pal)-specific binding agent, such as a polyclonal antibody, monoclonal antibody, antibody fragment or molecule that binds specifically to the Gram-negative Pal. For example, the urine can be exposed to an Enterobacteriaceae peptidoglycan associated lipoprotein (Pal)-specific binding agent, such as a polyclonal or monoclonal antibody, antibody fragment or molecule that binds specifically to Pal from Enterobacteriaceae. For example, the urine can be exposed to mouse monoclonal anti-Pal antibody (6D7), which binds specifically to Pal from E. coli, and also cross-reacts (binds) with Pal from any Enterobacteriaceae.

Binding of Gram-negative peptidoglycan associated lipoprotein (Pal) to the binding agent can be detected with a known output or measurement. Detection methods include fluorescence, a change in color, a change in light scattering, or an enzyme assay that is sensitive to the binding of Pal to its binding agent. For example, a strip will change colors or another visual output will appear when Pal is present in the urine sample. A test may be designed to detect a certain level of Gram-negative Pal in the urine above a specific threshold concentration.

Alternatively, the specific Pal levels (protein concentration) may be measured using a more complex test. In that case, Pal levels may be normalized to a standard urine component, such as creatinine. Such a normalization factor would be preferred since each person's urine is different and may be more or less diluted with water. By quantifying the creatinine levels in the urine, a specific Pal concentration can be determined and normalized to that creatinine concentration. The quantitative Pal levels may be measured to determine the severity of the patient's sepsis/bacteremia or to track the patient's disease and recovery after the initial diagnosis.

An embodiment of the disclosure includes a point-of-care (POC) assay, similar to a pregnancy test, which detects the presence of Pal in the urine of the patient. The assay can be performed with a Pal antibody or Pal-specific binding agent coated or bound to a strip. When Pal is present in the urine, the Pal would bind to the strip, resulting in a color change or some sort of visual change in the strip, notifying the clinician of the presence of Pal in the patient's urine.

An embodiment of the disclosure includes a device that can be used as a point of care for sepsis diagnosis, which can be similar to a dipstick pregnancy test. Components of the device may include a test window and optionally, a control window; an absorbent strip; an immunoassay strip, which contains the Pal-specific binding agent and optionally, a second binding agent to detect creatinine or another control; a container that houses the strips; and a cap to cover the absorbent strip. The device can be stored in a sealed package.

A more complex method/device could be used to quantify Pal levels in a patient's urine. The device would include the creation of a standard curve using samples of Pal protein at known concentrations; an output (such as absorbance of light) that correlates to protein concentration; a similar measurement performed on patient urine, as well as a control protein sample; and a calculation, which uses the standard curve and the urine sample measurements to estimate the actual Pal concentration in the urine sample.

The components of a kit that can be used as a point of care for sepsis diagnosis would be the device as described above, with the addition of a sterile wipe and cup for clean catch urine collection and a syringe and filter for optional removal of whole bacterial cells from the urine.

Methods in accordance with the present disclosure to detect Gram-negative sepsis in human patients preferably would be able to detect Pal from one or more Gram-negative bacteria(um) in the urine of those patients at an early stage of sepsis. Pal release is known to be enhanced by certain antibiotics, but a background level of Pal is released in the presence of human sera without antibiotics; therefore the Pal levels detected in urine should, in general, correlate with the amount of bacteria in the blood.

A more complex test (e.g., an enzyme-linked immunosorbent assay-ELISA) could be used to quantify the Pal in urine and therefore help determine the severity of sepsis and/or “track” the progression of the disease and/or recovery of the patient.

The present concept uses peptidoglycan associated lipoprotein (Pal) from Gram-negative bacteria as a urine biomarker for sepsis or bacteremia. Pal is commonly found in Gram-negative bacteria and is localized to the outer membrane via its lipid anchor (which embeds itself in the outer membrane of the bacterium). Much is known about Pal from E. coli, which is the most commonly studied Enterobacteriaceae. E. coli Pal is known to be shed from E. coli under certain conditions, such as in the presence of human blood or sera or when the bacteria are exposed to antibiotics. During an E. coli infection, Pal is released from the bacterium. When Pal is released by E. coli in the blood of human patients, Pal may also be filtered into urine. Since urine contains far fewer proteins than human serum, low levels of Pal in urine are detectable.

Anti-Pal or another molecule that binds specifically to Pal is used to detect Pal that is shed into the urine of patients with E. coli sepsis. Because E. coli Pal is highly similar in structure to Pal from other Enterobacteriaceae, an antibody/molecule used to detect E. coli Pal would likely cross-react with Pal from any Enterobacteriaceae. It is important to note that E. coli is a commensal organism found in the intestines of healthy humans. This E. coli, as part of the healthy flora, does not shed Pal that is detectable in the urine of healthy humans.

The present methods are the first known to be able to detect Pal in the urine of sepsis patients. Most studies on Pal/sepsis have focused on Pal's role in sepsis and its potential role as a therapeutic. The inventors had access to sepsis patient urine and Pal monoclonal antibody, and therefore were able to confirm Pal's presence in the urine of sepsis patients.

Successful diagnosis of sepsis can be a difficult task, as no single method currently provides a definitive diagnosis. As described above, in many cases, patient outcomes are greatly dependent on efficient and early diagnosis of the disease. The method provides a quick and reliable alternative for sepsis diagnosis. The Pal detection test could also be employed in combination with the current sepsis diagnostic tests in order to create a more accurate and comprehensive diagnosis protocol.

The present technology allows for point of care, low-cost, quick, reliable diagnosis of sepsis. This process also removes the need for a blood draw, which can be challenging in elderly or very young patients. Many sepsis patients already have catheters, making the collection of urine even more efficient. Since the test requires urine and not blood, this test greatly reduces the risk of contracting blood borne diseases for healthcare professionals.

The disclosure will be further illustrated with reference to the following specific examples. It is understood that these examples are given by way of illustration and are not meant to limit the disclosure or the claims to follow.

### Example

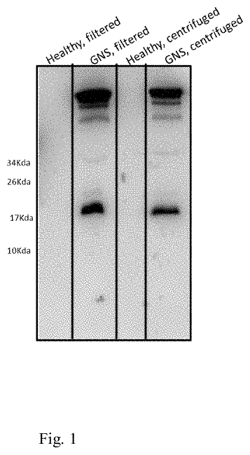

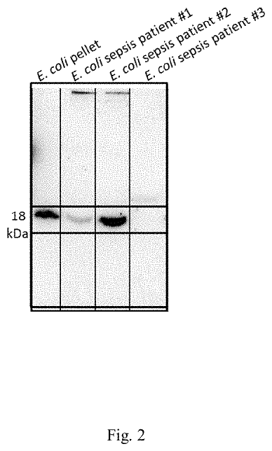

This example detected recombinant Pal (genetically modified to remove the N-terminal lipid attachment) spiked into healthy urine at levels as low as 0.2 ng/μl, without any purification step. This procedure also detected (with monoclonal anti-Pal) a putative Pal band in the urine of patients with diagnosed E. coli sepsis (FIG. 1). FIG. 1 shows a Pal immunoblot of healthy urine and urine from a patient with E. coli sepsis. The first two samples were syringe filtered to remove any potential whole bacterial cells, and the last two samples were gently centrifuged (5000×g) to remove any whole bacterial cells. Bands were detected at the same MW of native Pal. The same protein band was not detected in urine from healthy donors or elderly patients with acute inflammation, but no sepsis or urinary tract infections (UTI) (not shown). A similar 18-kDa protein was detected in additional urine samples from E. coli sepsis patients using monoclonal Pal antibody (FIG. 2). In FIG. 2 an anti-Pal immunoblot detects proteins in urine samples from three E. coli sepsis patients. All urine samples were filtered (with a 0.2 μm filter) to remove intact cells. The ˜16 kDa bands were detected in the urine of Patients #1 and #2 using monoclonal anti-Pal. Also considered was the possibility that this procedure was detecting Pal from a UTI. However, as seen in FIG. 2, Patient #3 was diagnosed with a UTI, but Pal was not observed in that sample. Putative Pal bands were observed in Patients #1 and #2, only one of whom was diagnosed with a UTI, suggesting that the putative Pal bands were not associated with UTI.

All urine samples were obtained from Rochester General Hospital; patients were confirmed GNS patients unless otherwise noted. The urine was kept at 4° C. until prepared, as described below. The urine was either filtered (0.2 μm filter) or gently centrifuged (5000×g) to remove intact cells. The samples were then combined at a 1:1 ratio with 2× Sample Buffer (recipe: 0.12 M Tris/HCl pH 6.8, 4% SDS, 20% glycerol, 0.01% bromophenol blue) and boiled for 10 minutes. The urine samples were then separated via sodium dodecyl sulfate polyacrylamide gel electrophoresis (SDS-PAGE) (10% gel). Proteins were transferred to a Nitrocellulose membrane (Pierce) and blocked with 5% milk in Tris buffered saline (TBS). The membrane was incubated with monoclonal anti-Pal at a 1:4000 dilution in 1% milk and TBS and then horseradish peroxidase (HRP) conjugated goat anti-mouse IgG (Bethyl Laboratories) at a 1:12,000 dilution in 1% milk and TBST (TBS with 0.05% Tween-20). The membrane was washed with TBS or TBST between antibody incubations. The blot was visualized using the Lumiglo Reserve HRP chemiluminscent substrate kit (KPL) according to the manufacturer's instructions.

In summary, the present experimental data suggest that Gram-negative Pal can act as a biomarker for Gram-negative bacterial sepsis in the urine of human patients.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed:

1. A method for detecting septic or bacteremic levels of Gram-negative bacteria in a patient, comprising:
obtaining the urine of a human patient;
exposing the urine to a Gram-negative peptidoglycan associated lipoprotein specific binding agent; and
detecting the Gram-negative peptidoglycan associated lipoprotein bound to the binding agent.

2. The method of claim 1, wherein the binding agent comprises a polyclonal antibody, monoclonal antibody, antibody fragment or molecule that binds specifically to a peptidoglycan associated lipoprotein from Gram-negative bacteria.

3. The method of claim 1, wherein the Gram-negative peptidoglycan associated lipoprotein comprises Enterobacteriaceae peptidoglycan associated lipoprotein.

4. The method of claim 1, wherein the Gram-negative peptidoglycan associated lipoprotein specific binding agent comprises Enterobacteriaceae peptidoglycan associated lipoprotein specific binding agent.

5. The method of claim 4, wherein the Enterobacteriaceae peptidoglycan associated lipoprotein specific binding agent comprises mouse monoclonal anti-Pal antibody (6D7).

6. The method of claim 1, wherein detecting peptidoglycan associated lipoprotein bound to the binding agent comprises a visual determination.

7. The method of claim 1, further comprising filtering out whole cell bacteria from the urine prior to exposing the urine to the binding agent.

8. A device comprising:
a test window and optionally, a control window;
an absorbent strip;
an immunoassay strip, comprising a Gram-negative peptidoglycan associated lipoprotein-specific binding agent and optionally, a control-specific binding agent;
a container housing the absorbent and immunoassay strips; and
a cap covering the absorbent strip.

9. The device of claim 8, further comprising a sealed package enclosing the device.

10. The device of claim 8, wherein the control-specific binding agent comprises a creatinine-specific binding agent.

11. The device of claim 8, further comprising:
a standard curve comprising samples of peptidoglycan associated lipoprotein protein at known concentrations;
an output that correlates to protein concentration;
a similar measurement performed on patient urine, as well as a control protein sample; and
a calculation, which uses the standard curve and the urine sample measurements to estimate the peptidoglycan associated lipoprotein concentration in the urine sample.

12. The device of claim 11, wherein the output comprises an absorbance of light.

13. A kit comprising:
a device comprising:
a test window and optionally, a control window,
an absorbent strip,
an immunoassay strip, comprising a Gram-negative peptidoglycan associated lipoprotein-specific binding agent and optionally, a control-specific binding agent,
a container housing the absorbent and immunoassay strips, and
a cap covering the absorbent strip;
a sterile wipe and clean catch urine collection cup; and
a syringe and filter for optional removal of whole bacterial cells from the urine.

14. The kit of claim 13, wherein the device further comprises:
a standard curve comprising samples of peptidoglycan associated lipoprotein protein at known concentrations;
an output that correlates to protein concentration;
a similar measurement performed on patient urine, as well as a control protein sample; and
a calculation, which uses the standard curve and the urine sample measurements to estimate the peptidoglycan associated lipoprotein concentration in the urine sample.

15. The kit of claim 14, wherein the output comprises an absorbance of light.
